SEQUENCE LISTING (7 sequences)

SEQ ID NO: 1 - DNA, 1094 bp, Artificial Sequence
  Fully synthetic
gtcaagtccaacagcagtgaaacaacaccaatacggcccacggccaaactgagcatttcaacccgttatcaagagggtgcattcacccgggggggccacagggaccaagggcccttccaagatcaactttctgtggctcatgaaagacgctgccccttccaccaatgctctgtgcgggatcgggtctggagagctggtcccgctgcaggaagaggcgaaagaggaagagaaagcaggcgtgtgtgcccgctccgtgcacttgagggaaattgcacagccctccagcggaaatcttggagcagccctcaaagacgcaaaagcaacgcatcctcctagaagcctaaacgtttggaacgcaggttgccttacttctctctctctttttgttttttgttttttcgttgtgatttctgctttctaaatcgcacacctgcaatggacgggagctgaaggccctttcttactgacaagggacagaagccgcaaatctccctgatgccgactcattctgcaacctgtcacctgcatcaaagagccggagaggcaagtctggacagaactggctgctcagagccctgtgggagcttcctcccagagagaacgaggaggctggggaccgagagaaggggacagagacagaggggagggaaagtcagcaccagggagagggaaaaaagagccacctggaacagcagacgcgcagacaccaggacgtggagaggacaagccagggaaaacgtagttttctatgctgacacgtcagcaagcgtatcatctgggtgtatgcatggctgtgacagaggcaccattactcatgcttgagtgaaagaaataagacccatccagtgcttaggcgctattctcgctggcattgattctcacaaaggcagcacctgcatgctttgtgcgtcttgatgcagtgacgtcaccctctcacttgggaaacctaaaccaccaaatcccctgcatcccacttcacttccctccaccattatggttccatttaaaagggcaaaatatgaactgcttgacctgtggtaccttaaggcacaatttgagtctaatggaataaatgtgttcatacaagtcctgag

SEQ ID NO: 2 - DNA, 18 bp, Artificial Sequence
  Fully synthetic
tcagcaagcgtatcatct

SEQ ID NO: 3 - DNA, 20 bp, Artificial Sequence
  Fully synthetic
gggtcttatttctttcactc

SEQ ID NO: 4 - DNA, 18 bp, Artificial Sequence
  Fully synthetic
gctgctgctcatactact

SEQ ID NO: 5 - DNA, 18 bp, Artificial Sequence
  Fully synthetic
cacttctggcttcttcct

SEQ ID NO: 6 - DNA, 18 bp, Artificial Sequence
  Fully synthetic
ataagagttcagcgacat

SEQ ID NO: 7 - DNA, 18 bp, Artificial Sequence
  Fully synthetic
gtggttattgaggagtag

(19) United States
(12) Patent Application Publication
(10) Pub. No.: US 2019/0203272 A1
(43) Pub. Date: Jul. 4, 2019

(54) USE OF BRCA1 AND/OR JAML GENES IN PREDICTING INTRAMUSCULAR FAT CONTENT OF PORK AND IN SELECTIVE BREEDING OF PIGS

(71) Applicants: Feng SHAO, (US); Xiangyang MIAO, (US)

(72) Inventors: Xiangyang MIAO, Beijing (CN); Wanlong HUANG, Beijing (CN); Ai LI, Beijing (CN); Lingli XIE, Beijing (CN); Xiuxiu ZHANG, Beijing (CN)

(21) Appl. No.: 16/234,540

(22) Filed: Dec. 27, 2018

(30) Foreign Application Priority Data
Dec. 28, 2017 (CN) 201711464765.0

(51) Int. Cl. C12Q 1/6827; C12Q 1/6816; C12Q 1/686; C12Q 1/6851; A01K 11/00

(52) U.S. Cl. CPC C12Q 1/6827; C12Q 1/6816; A01K 2227/108; C12Q 1/6851; A01K 11/00; C12Q 1/686

(57) ABSTRACT

A method of predicting the variation of porcine intramuscular fat profile. The method includes detecting a BRCA1 gene and/or a JAML gene, or an expression product thereof. The disclosure also provides a method of selecting and breeding pigs. The method includes detecting a BRCA1 gene and/or a JAML gene, or an expression product thereof.

### CROSS-REFERENCE TO RELATED APPLICATIONS

Pursuant to 35 U.S.C. § 119 and the Paris Convention Treaty, this application claims foreign priority to Chinese Patent Application No. 201711464765.0 filed Dec. 28, 2017, the contents of which, including any intervening amendments thereto, are incorporated herein by reference. Inquiries from the public to applicants or assignees concerning this document or the related applications should be directed to: Matthias Scholl PC., Attn.: Dr. Matthias Scholl Esq., 245 First Street, 18th Floor, Cambridge, Mass. 02142.

### BACKGROUND

The disclosure relates to the use of a BRCA1 gene and/or a JAML gene in predicting variation of porcine intramuscular fat profile and/or selective breeding of pigs.

### SUMMARY

The disclosure provides a method of predicting variation of porcine intramuscular fat profile, the method comprising detecting a BRCA1 gene and/or a JAML gene, or an expression product thereof.

The disclosure further provides a method of selecting and breeding pigs, the method comprising detecting a BRCA1 gene and/or a JAML gene, or an expression product thereof.

According to the disclosure, the candidate BRCA1 and JAML genes are selected by high-throughput sequencing in combination with bioinformatics. The method detects whether the genes are related to pork intramuscular fat.

The expressions of the BRCA1 gene and/or the JAML gene are detected using sequencing technique, nucleic acid hybridization technique, or nucleic acid amplification technique.

The nucleic acid hybridization technique comprises hybridizing a probe to a nucleic acid sequence of the BRCA1 gene and/or the JAML gene; the nucleic acid amplification technique comprises amplifying the BRCA1 gene and/or the JAML gene using a pair of primers.

The expressions of the BRCA1 gene and/or the JAML gene are detected using immunization means.

The expressions of the BRCA1 gene and/or the JAML gene are detected using an ELISA detection kit and/or a colloidal gold detection kit.

The disclosure further provides a long non-coding RNA (lncRNA) XLOC_011001, having more than 90% sequence homology with SEQ ID NO. 1.

The lncRNA has more than 95% sequence homology with SEQ ID NO. 1. Particularly, the lncRNA has the sequence shown in SEQ ID NO. 1.

Also, provided is a method of using the long non-coding RNA (lncRNA) in predicting variation of porcine intramuscular fat profile, in selecting and breeding pigs, or in preparation of a kit adapted to detect variation of porcine intramuscular fat profile, the method comprising detecting the expression of the lncRNA.

Also, provided is a kit adapted to detect variation of porcine intramuscular fat profile, the kit comprising a pair of primers for nucleic acid amplification, and the sequences of the pair of primers are represented by SEQ ID NO. 2 and SEQ ID NO. 3.

Also, provided is a method of using the kit in predicting variation of porcine intramuscular fat profile or in selecting and breeding pigs, the method comprising detecting a BRCA1 gene and/or a JAML gene, or an expression product thereof.

The sample detected by the kit is porcine tissue, particularly, intramuscular adipose tissue.

The methods of the disclosure include but are not limited to any particular variants of XLOC_011001. In some embodiments, the variants contain at least 85% of the same or similar cDNA sequence of XLOC_011001, e.g., at least 90%, 91%, 92%, 93%, 94%, 95%, 96%, 97%, 98% or at least 99% of the sequences listed above are of the same or similar cDNA sequences.

The term “homology”, as used herein, primarily refers to homology in sequence, which means two or more proteins or DNA sequences have the same sources. Homologous sequences generally have similar functions. The homology between protein and DNA is often determined by the similarity of their sequences. The similarity refers to the proportion of the same DNA base or amino acid residue sequence between the detection sequence and the target sequence in the sequence alignment. In general, when the similarity is higher than 50%, it is often assumed that the detection sequence and the target sequence may be homologous; when the similarity is less than 20%, the homology cannot be determined.

The nucleic acid amplification technique includes Polymerase Chain Reaction (PCR), Reverse Transcription Polymerase Chain Reaction (RT-PCR), Transcription-Mediated Amplification (TMA), Ligase Chain Reaction (LCR), Strand Displacement Amplification (SDA) and Nucleic Acid Sequence Based Amplification (NASBA). Among them, the PCR requires reverse transcription of RNA into DNA (RT-PCR) prior to amplification, while the TMA and NASBA directly amplify RNA.

Typically, PCR uses multiple cycles of denaturation, annealing of primer pairs and opposite strands, and primer extension to exponentially increase the copy number of the target nucleic acid sequence; RT-PCR uses Reverse Transcriptase (RT) for preparation of complementary DNA (cDNA) from mRNA, and then the cDNA is amplified through PCR to produce multiple copies of the DNA; TMA autocatalytically synthesizes multiple copies of the target nucleic acid sequence under roughly constant temperature, ionic strength, and pH value; the multiple copies of the RNA of the target sequence autocatalytically synthesize additional copies, TMA arbitrarily blocks, terminates, modifies part of sequences to improve the sensitivity and accuracy of the amplification process; LCR uses the two sets of complementary DNA oligonucleotides that hybridize with the adjacent regions of the target nucleic acid. DNA oligonucleotides are covalently linked by DNA ligase in multiple cycles of heat denaturation, hybridization, and ligation to generate a detectable double-stranded oligonucleotide product; SDA is cyclically performed as follows: the annealing of the primer sequence pair from the opposite strand of the target sequence; the primer extension in the presence of dNTPαS to generate a double-strand hemiphosphorothioated product; the endonuclease mediated cleavage at a semi-modified restriction endonuclease recognition site, and the polymerase-mediated primer extension from the 3′-end of the incision to produce a strand adapted to replace the existing strand for next cycle of annealing, cutting and displacement of the primer, thus geometrically amplifying the product.

The “Probe” as used in this disclosure refers to a molecule capable of binding to a sequence or subsequence or other portion of another molecule. Unless otherwise stated, the term “probe” generally refers to a polynucleotide probe capable of binding to another polynucleotide (often referred to as a “target polynucleotide”) by complementary base pairing. Depending on the stringency of the hybridization conditions, the probe binds to a target polynucleotide that lacks complete sequence complementarity to the probe. The probe can be labeled directly or indirectly, and includes the primers. The hybridization methods include, but are not limited to, solution phase, solid phase, mixed phase or in-situ hybridization assay.

The probe has a base sequence complementary to a specific base sequence of a target gene, where the term “complementary” is not necessarily completely complementary as long as it can be hybridized. These polynucleotides usually have more than 80% of homology with respect to the specific base sequence, particularly, more than 90%, more particularly, more than 95%, and further particularly, 100% homology. These probes may be DNA or RNA, and may also be polynucleotides obtained by substituting part or all sequences of the DNA or RNA by artificial nucleic acids such as PNA (Polyamide Nucleic Acid), LNA (Locked Nucleic Acid, Bridged Nucleic Acid), ENA (2′-O, 4′-C-Ethylene-bridged nucleic acids), GNA (Glycerol nucleic acid), TNA (threose nucleic acid).

The term “hybridization” in this disclosure refers to the pairing of complementary nucleic acids. The strength of hybridization and hybridization (i.e., the strength of association between nucleic acids) is affected by the following factors such as the degree of complementarity between nucleic acids, the stringency of the reaction conditions, the G:C ratio and the melting temperature of the formed hybrid. The single molecule containing complementary nucleic acid pairs within its structure is referred to as “self-hybridizing.”

The Nucleic acid hybridization techniques as used in this disclosure include, but are not limited to, In-situ Hybridization (ISH), Microarrays, and Southern or Northern blots. The In-situ Hybridization (ISH) uses a labeled complementary DNA or RNA strand as a probe to localize a specific DNA or RNA sequence in a portion or section of the tissue, or the entire tissue (Tissue-wide ISH embedding) if the tissue is small enough. DNA ISH can determine the structure of a chromosome. RNA ISH is used to measure and localize mRNA and other transcripts (e. g., ncRNA) in tissue sections or in whole tissues. Sample cells and tissues are often preprocessed to immobilize the target transcript in situ and increase the entry of the probe. The probe hybridizes with the target sequence at elevated temperatures and then the excess probe is washed away. The base-labeled probes in tissues labeled with radioactive, fluorescence or antigen labels are localized and quantified using autoradiography, fluorescence microscopy, or immunohistochemistry, respectively. ISH can also use two or more probes labeled with radioactive or other non-radioactive labels to simultaneously detect two or more transcripts.

### BRIEF DESCRIPTION OF THE DRAWINGS

FIG. 1 is a distribution map of differentially expressed genes in intramuscular fat;

FIG. 2 is a diagram showing the results of qRT-PCR verification of differentially expressed genes;

FIG. 3 is a diagram showing the results of qRT-PCR verification of the differentially expressed gene XLOC_011001; and

FIG. 4 is a graph showing the results of qRT-PCR verification of BRCA1 and JAML genes.

### DETAILED DESCRIPTION

### Example 1: Sample Collection, Preparation and Experimental Design

The experimental pigs, including Large white pig (D_JN) and Laiwu pig (L_JN), were slaughtered, and the intramuscular adipose tissues of the longissimus dorsi were collected, cut into small pieces, placed in a 5 mL cryotube, frozen in liquid nitrogen, and then stored in a 0° C. refrigerator for extraction of total RNA. The lncRNAs in the intramuscular adipose tissues of Large white pig (D_JN) and Laiwu pig (L_JN) were identified, and the gene expressions of the intramuscular adipose tissues were analyzed. The Large white pig (D_JN) and the Laiwu pig (L_JN) were both provided with three experimental groups, and each group was provided with three samples.

### Example 2: Extraction and Quality Control of Total RNA of Samples

Equal amounts of cryopreserved adipose tissue samples were taken. The total RNA of each adipose tissue sample was extracted using a mirVana™ RNA extraction kit according to the instruction manual. The separated total RNAs were stored in a −80° C. refrigerator. The concentration of the RNAs and the OD 260 nm/OD 280 nm values were measured using a NanoDrop 2000 spectrophotometer and controlled at 1.9-2.1. The quality of total RNA was evaluated with Bioanalyzer 2100, and the RNA integrity number (RIN) was controlled>=7 and 28S/18S>=0.7. The potential genomic DNA contamination was eliminated using RNase-free DNase I.

### Example 3: cDNA Library Construction and RNA Sequencing

The strand-specific cDNA library

(1) Removal of rRNA with Ribo-zero kit

(2) RNA fragmentation

(3) Double-stranded cDNA synthesis and purification

(4) End repair, adding nucleotide A

(5) Sequencing Connector Connection

(6) Enrichment and purification of DNA fragments

(7) Library Quality Inspection

(8) 6 cDNA libraries were established, namely D_JN_1, D_JN_2, D_JN_3 (cDNA library of intramuscular adipose tissue of Large white pig) and L_JN_1, L_JN_2, L_JN_3 (cDNA library of intramuscular adipose tissue of Laiwu pig).

RNA-Seq (Illumina Sequence)

The libraries were tested qualified. The Illumina Hi Seq™ 2500 sequencing platform was used to analyze the cDNA libraries by using the Paired-end Sequence, and the off-line data was the raw reads.

### Example 4: Quality Control and Filtering of Raw Reads

Raw reads contain low quality and contaminated sequences that must be filtered out to ensure the accuracy and reliability of the subsequent bioinformatics analysis. The quality control of the raw reads was conducted using Cutadapt (v1.12) and FASTX_toolkit (v0.1.14) software. The subsequent analysis was all based on the clean reads. The operations were as follows:

(1) removing the reads having an adapter sequence;

(2) filtering out the reads containing no more than 10% of the base (N);

(3) removing the low-quality reads in which the bases having a mass value Q<20 accounted for more than 15% of the total bases;

The results are shown in Table 1. Through the quality control, about 90 million clean reads were obtained in each sample, and contain around 95% of the bases having Q-score≥30. The GC base content was about 50%. This indicated that the sequencing data results were reliable, and can be used for further analysis.

TABLE 1

| Samples | Raw reads | Clean reads | Valid ratio(base) | Q30(%) | GC content(%) |
|---|---|---|---|---|---|
| Sample_D_JN_1 | 94261614 | 90180702 | 95.61% | 94.48% | 51.00% |
| Sample_D_JN_2 | 94457608 | 90392480 | 95.63% | 94.48% | 49.50% |
| Sample_D_JN_3 | 94739548 | 91093142 | 96.09% | 94.74% | 53.50% |
| Sample_L_JN_1 | 92489412 | 88890352 | 96.05% | 95.30% | 50.00% |
| Sample_L_JN_2 | 92272792 | 89083366 | 96.48% | 95.60% | 51.00% |
| Sample_L_JN_3 | 95371148 | 91659768 | 96.04% | 95.26% | 49.00% |

### Example 5: Reference Genome Alignment and Transcript Splicing

The clean reads were aligned to the reference genome to locate the reads. Download the pig's reference genome Sscrofa 10.2 and the annotation file Sscrofa 10.2.87.chr.gtf from the Ensembl database. Then, use the bowtie software (v2.2.5) bowtie-build to establish the reference genome index, and use TopHat (v2.0.12) software to compare the clean reads obtained from each sample to the reference genome. The mismatch was set as 2, and others adopted default parameters.

To predict new transcripts, the transcripts required reconstruction and assembly. Inputting the sequence alignment file accepted_hit. bam obtained after aligning the clean reads to the genome using the TopHat2 software. Using Cufflinks (v2.1.1) software to assemble transcripts for each sample to get the annotation file transcript. gtf. Using Cuffmerge to assemble the gtf files of 12 samples and merge them to generate the annotation file merged transcript. gtf. Using Cuffcompare to compare merged transcript. gtf with the reference annotation file Sscrofal 0.2.87. chr. gtf one by one, selecting the transcripts that matched or were similar to known ncRNAs, mRNAs, etc., locating the location information of the transcripts, and predicting the potential new mRNA and lncRNA.

Results: Clean reads were compared to the pig's reference genome using bioinformatics software. The results are shown in Table 2.

TABLE 2

| | Mapped types | | | |
|---|---|---|---|---|
| | | | Multiple mapped | Uniquely mapped |
| Samples | Total reads | Total Mapped reads | reads | reads |
| Sample_D_JN_1 | 90180702 | 68721069(76.2%) | 11568665(12.83%) | 57152404(63.38%) |
| Sample_D_JN_2 | 90392480 | 90392480(76.31%) | 11136845(12.32%) | 57838075(63.99%) |
| Sample_D_JN_3 | 91093142 | 69180297(75.94%) | 14614985(16.04%) | 54565312(59.9%)) |
| Sample_L_JN_1 | 88890352 | 65333853(73.5%) | 11406632(12.83%) | 53927221(60.67%) |
| Sample_L_JN_2 | 89083366 | 65710645(73.76%) | 10421104(11.7%) | 55289541(62.06%) |
| Sample_L_JN_3 | 91659768 | 67630486(73.78%) | 11270084(12.3%) | 56360402(61.49%) |

### Example 6: Analysis of Alternative Splicing Events

Using ASprofile (v1.0) software to analyze the assembly files of each sample, and statistically classifying the variable splicing events. According to the structure of the exons and the status of the intron retention, the alternative splicing (AS) events were defined into 12 different categories, including TSS, TTS, SKIP, XSKIP, MSKIP, XMSKIP, IR, XIR, MIR, XMIR, AE, XAE.

### Example 7: Selection and Identification of Potential lncRNA

LncRNA is a class of RNA that is longer than 200 bp and does not encode proteins. The potential lncRNA can be identified based on the two main characteristics, and comprises intergenic lncRNA (lincRNA), intronic lncRNA, sense lncRNA and antisense lncRNA. The operations are as follows:

(1) Exon number and transcript length selection: the threshold was exon number≥2, length>200 bp, and the single exon transcript of low-confidence was filtered out.

(2) Coding potential selection: for the above selected transcripts, using software PLEK (Li et al., 2014), CNCI (Sun et al., 2013b), CPC (Kong et al., 2007), Pfam (Finn et al., 2014) to predict their protein-coding potential, and analyzing the intersection to obtain the lncRNA. PLEK was based on optimized k-mer strategy, threshold score<0. CNCI was based on triplet spectrum of sequence adjacent nucleotides, threshold score<0. CPC was based on the sequence characteristics of the open reading frame of transcripts, and the sequence characteristics were also compared with UniProt reference database BLASTX, threshold score<0. Pfam was a protein family database, and can homologously match the transcript coding frame with the database; the transcript that was not matched was lncRNA.

(3) The identification of lncRNA: ALB (A Domestic-Animal Long Noncoding RNA Database) was a database of livestock animal lncRNA, and the candidate lncRNA was compared with the lncRNA in the database by BLASTN tool. The selected lncRNAs were identified strictly under the conditions of Identity=100%, mismatch=0, E-value<1e−10, and gap opening=0.

The classification, length distribution and number of exons of the lncRNAs were analyzed and compared with the identified mRNAs. The length distribution trend of the lncRNAs and the protein-encoding genes was roughly consistent, and the transcription density of the shorter mRNA was higher than that of the lncRNAs. The average length of the lncRNAs identified in this study was 2263 nt, and the average length of the mRNA was 2028 nt.

### Example 8: Analysis of Gene Differential Expression Between Different Samples

Building data sets of known mRNA, predicted new transcripts, and lncRNA. Using bowtie and eXpress software to compare and statistically analyze the read count of each transcript in each sample. The gene expression level was corrected using the fragment per kb per Million reads (FPKM) algorithm to eliminate the effects of sequencing depth, gene length and sample differences on the gene expression. The experiment was biologically repetitive. Using the R language package DESeq2 (Anders & Huber, 2010) to analyze the differential expression of genes (including lncRNA, mRNA) between different samples based on the negative binomial distribution. Benjamini-Hochberg algorithm was used for multiple hypothesis test calibration of P values to obtain a corrected P value (padj). The differentially expressed genes were selected under the condition of |log 2FoldChange|≥1 (L_JN vs D_JN) and padj≤0.05.

Based on the expression value FPKM of the transcripts, constructing a FPKM boxplot and density map to analyze the integral expression of the transcripts in the samples of different adipose tissues. The distribution of the expression of the transcripts in the intramuscular adipose tissue of the two breeds of pigs in the group were consistent. Compared with Laiwu pig, there were lower expression transcripts in the adipose tissue of Large white pig between groups. Analysis of the expression level of transcripts between samples shows that the experimental data basically meets the requirements. At the same time, the analysis of the expression levels of the identified lncRNA and mRNA shows that mRNA has a relatively high expression level, and the expression level of lncRNA is low. The FPKM values are mainly between (0-10), and the mRNA with FPKM value of 0-100 are evenly distributed.

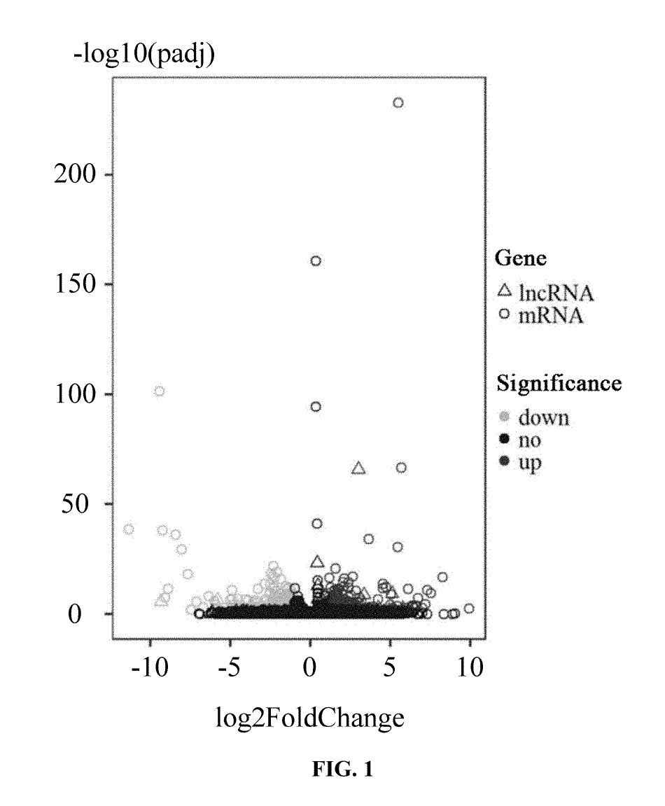

By differentially analyzing the intramuscular adipose tissue (L_JN vs D_JN) gene (FIG. 1), 56 differentially expressed lncRNAs (34 up-regulated, 22 down-regulated) and 715 differentially expressed mRNAs (371 up-regulated, 344 down-regulated) were identified, of which genes with more than 4 times of difference accounted for about 48.4 percent. Among them, differentially expressed genes represented by BRCA1 and JAML and differentially expressed long non-coding RNA represented by XLOC_011001 were selected for further analysis.

### Example 9: Differential Expression Gene GO and KEGG Pathway Enrichment Analysis

Gene Ontology (GO) is an international classification standard for gene function, which is composed of molecular function, biological process, and cellular component. The Pathway Enrichment Analysis can identify the major metabolic pathways and signaling pathways of differentially expressed genes. As the main public database of correlation, the KEGG (Kyoto Encyclopedia of Genes and Genomes) database is a main tool for metabolic analysis and regulation network research. To study the main biological functions of differentially expressed genes, the experiment used CluGO software to calculate the GO terms and signal pathways with significantly enriched differentially expressed genes based on hypergeometric distribution test. The P value (Q_value) was corrected by the Benjamini-Hochberg algorithm. The enrichment was significant when Q_value≤0.05.

513 database-annotated differentially expressed genes were identified in the intramuscular adipose tissue of Large white pig and Laiwu pig, and 210, 144, and 62 genes were enriched into one or multiple terms of the biological processes, molecular functions, and cellular components, respectively. A large number of GO terms closely related to lipid metabolism and deposition were significantly enriched. According to the bioprocess, more genes (≥15) were enriched in the lipid biosynthetic process, the lipid metabolic process, the cellular lipid metabolic process, response to lipid, MAPK cascade, positive regulation of MAPK cascade, and regulation of MAPK cascade. For the molecular functional part, it was only significantly enriched in the enzyme inhibitor activity. In the cellular components, it was significantly enriched in related GO terms such as extracellular matrix, axon. There were significant differences in intramuscular fat deposition between Large white pig and Laiwu pig. It was found by GO annotation that differentially expressed genes were significantly enriched in biological processes of lipid metabolism and cell differentiation, indicating the intramuscular fat deposition and metabolism were regulated by different genes.

### Example 10: Differentially Expressed Gene Protein—Protein Interaction Network (PPI Network) Analysis

The PPI network studies can reveal the function of proteins at the molecular level. Therefore, based on the interaction relationship in the STRING protein interaction database, the PPI network analysis of differentially expressed genes was carried out to further explore the complex interaction between the proteins encoded by differentially expressed genes in the muscle adipose tissue of Large white pig and Laiwu pig. The STRING database contains the breeding pig (Sus scrofa), and the interaction relationship of the differential gene set list can be extracted directly from the database. The Cytoscape software was used to visually analyze the data files of the obtained differential gene coded protein interaction network. In the protein interaction network diagram, the node represents the protein, the edge represents the interaction relationship between proteins, and the degree represents the number of proteins interacting with a specific node. The node size is proportional to the degree of the node. The color of the node indicates the log 2FoldChange value of the differentially expressed gene.

### Example 11: Prediction of Target Gene of Differentially Expressed lncRNA

As a non-coding RNA, lncRNA mainly functions in the regulation of target genes, including trans-regulation on the distant protein-coding genes. The genes with the same expression pattern have a strong functional correlation. Therefore, the target genes of the lncRNA can be identified by studying the co-expression and trans-regulation of the lncRNA and the mRNA.

The co-expression relationship between the lncRNA and the mRNA was analyzed by calculating the Pearson correlation coefficient (PCC) of the differential expression amount of the lncRNA and the mRNA. The co-expressed lncRNA-mRNA was selected with |PCC|>0.8 and P_value<0.05 as the threshold.

lncRNA trans target gene analysis: predict the trans target gene of differentially expressed lncRNA through the interaction relationship between the lncRNA and the mRNA. RNAplex software was used to calculate the binding free energy (Energy) between the lncRNA and the mRNA, and in combination with co-expression results, to identify the lncRNA trans target gene when Energy<−20 and |PCC|≥0.9.

The fat metabolism-related trans-target genes BRCA1 and JAML of the XLOC_011001 were found. The expression of the genes BRCA1 and JAML in the intramuscular fat of Laiwu pig was higher than that of Large white pig.

### Example 12: Fluorescence Quantitative PCR Verification of Differentially Expressed lncRNA

9 differentially expressed genes (4 lncRNAs and 5 mRNAs) were randomly selected from L_JN (Intramuscular tissue of Laiwu pigs) vs D_JN (intramuscular tissue of Large white pigs). Each gene was provided with 3 biological repetitions. The expression level of the genes was measured by qRT-PCR method with the actin beta (ACTB) gene as the internal reference. The cDNA template was synthesized from about 0.5 μg of RNA sample through reverse transcription by PCR System 9700 (Applied Biosystems, USA). The qRT-PCR analysis was performed using Green PCR Kit (Qiagen, Germany) 48011 Real-time PCR Instrument (Roche, Swiss).

The RNA to be tested was reversely transcribed into cDNA using HiScript II Q RT SuperMix for qPCR (+gDNA wiper) (Vazyme, R223-01).

(1) The total RNA sample stored in a −80° C. refrigerator was taken out, thawed at room temperature. The reverse transcription system in a 0.2 mL PCR tube was prepared as follows.

(2) Reverse Transcription System (10 μL): total RNA, 0.5 μg; 4×gDNA wiper Mix, 2 μL; adding Nuclease-free H₂O to 8 μL, Reaction conditions: 42° C. for 2 min. Adding 5×HiScript II Q RT SuperMix IIa, 2 μL, Reaction conditions: 25° C. for 10 min, 50° C. for 30 min, 85° C. for 5 min.

(3) Following the reverse transcription, the reverse transcription system was diluted with Nuclease-free H₂O to a total volume of 100 μL and stored at −20° C.

Real-Time PCR Reaction

(1) Compositions and volumes

TABLE 3
Components and volume in PCR

| Components | Volume (μL) |
|---|---|
| 2 × QuantiFast SYBR | 5 |
| Green PCR Master Mix | |
| Forward primer (10 μM) | 0.2 |
| Reverse primer (10 μM) | 0.2 |
| Nuclease-free H₂O | 3.6 |
| cDNA | 1 |
| Total | 10 |

(2) Cycling conditions

TABLE 4
Cycling conditions of PCR

| Steps | Temperature (° C.) | Time | Cycle number |
|---|---|---|---|
| Initial denaturation | 95 | 10 min | 1 |
| Denaturation | 95 | 10 s | 40 |
| Annealing | 60 | 30 s | |
| Dissociation | 60 to 97 | | 1 |

(3) The compositions were uniformly mixed, centrifuged, distributed on a 384-well plate, and transferred to LightCycler® 480 II Real-time PCR Instrument (Roche, Swiss) for qRT-PCR reaction and analysis.

The relative expression of genes in each group of samples was calculated by 2-ΔΔ Ct method. The relative expression amount was statistically analyzed by t-test. The data were expressed as mean±standard deviation (Mean±SD), where P<0.05 indicated that the difference was significant.

FASN, XLOC_002561, XLOC_053194, CD36, and MAP3K4 were significantly up-regulated in the intramuscular fat of Large white pigs.

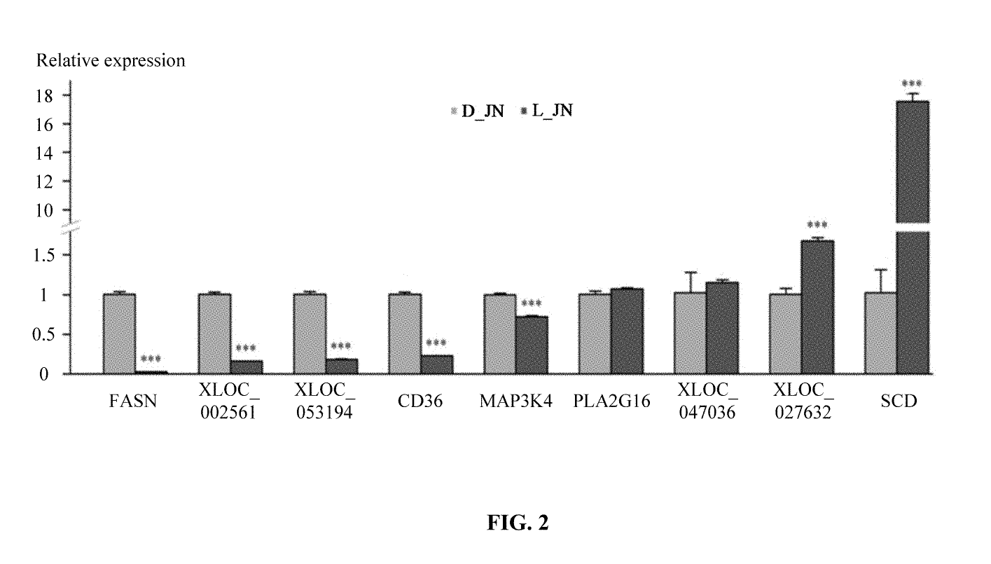

The expression of XLOC_027632 and SCD were significantly up-regulated in the intramuscular fat of Laiwu pig (FIG. 2). The above results were consistent with the sequencing results, indicating that the sequencing results were reliable.

10 intramuscular tissue samples of Large white pigs and 10 intramuscular tissue samples of Laiwu pig were randomly selected for the fluorescence quantitative verification of the candidate genes XLOC_011001, BRCA1 (XM_021066931.1), JAML (NM_001244730.1). The operations were the same as above.

Primer design:
XLOC_011001:
Upstream primer:
(SEQ ID NO. 2)
5′-TCAGCAAGCGTATCATCT-3′
Downstream primer:
(SEQ ID NO. 3)
5′-GGGTCTTATTTCTTTCACTC-3′
BRCA1 gene:
Upstream primer:
(SEQ ID NO. 4)
5′-GCTGCTGCTCATACTACT-3′
Downstream primer:
(SEQ ID NO. 5)
5′-CACTTCTGGCTTCTTCCT-3′
JAML gene:
Upstream primer:
(SEQ ID NO. 6)
5′-ATAAGAGTTCAGCGACAT-3′
Downstream primer:
(SEQ ID NO. 7)
5′-GTGGTTATTGAGGAGTAG-3′

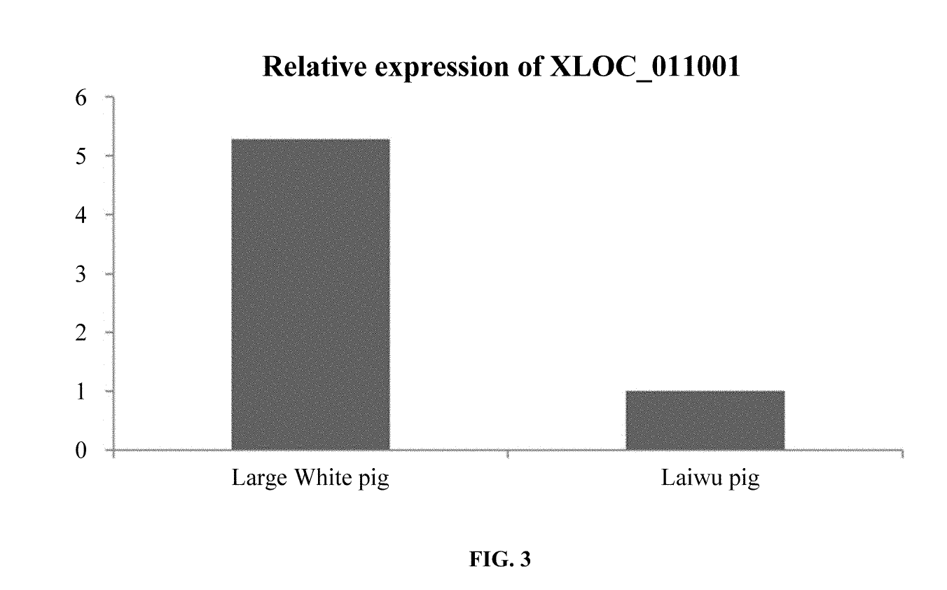

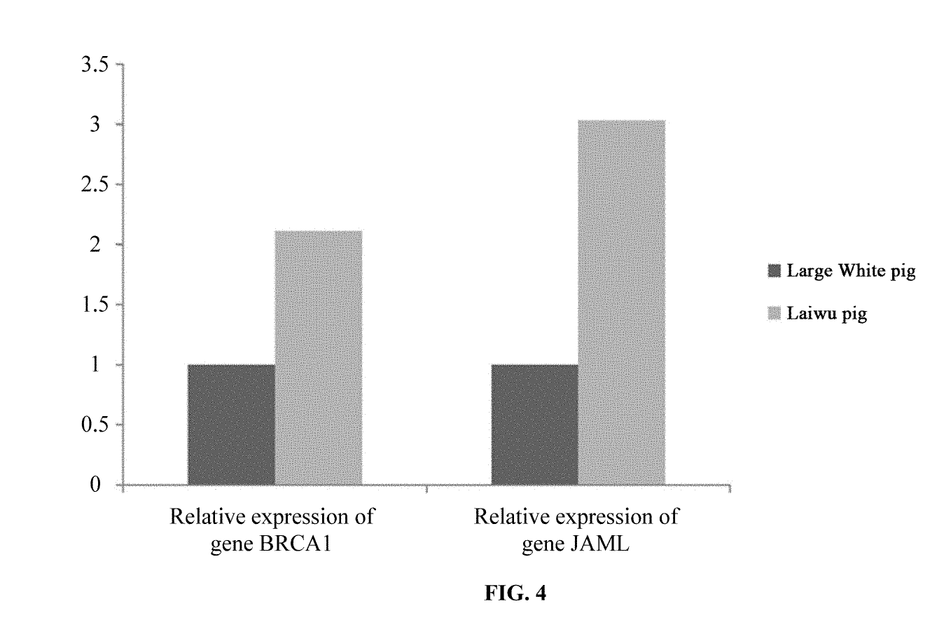

The results are shown in FIG. 3 and FIG. 4. The expression of XLOC_011001 in the intramuscular fat of Laiwu pig is about 5 times that of the intramuscular adipose tissue of Large white pig. The BRCA1 gene in the intramuscular fat of Laiwu pig is about twice as much as that in the intramuscular adipose tissue of Large white pig. The JAML gene in the intramuscular fat of Laiwu pig is about 3 times as much as that in the intramuscular adipose tissue of Large white pig.

It will be obvious to those skilled in the art that changes and modifications may be made, and therefore, the aim in the appended claims is to cover all such changes and modifications.

What is claimed is:

1. A method, comprising detecting a BRCA1 gene and/or a JAML gene, or an expression product thereof, in an intramuscular porcine adipose tissue.

2. The method of claim 1, wherein expressions of the BRCA1 gene and/or the JAML gene are detected using a sequencing technique, a nucleic acid hybridization technique, or a nucleic acid amplification technique.

3. The method of claim 2, wherein the nucleic acid hybridization technique comprises hybridizing a probe to a nucleic acid sequence of the BRCA1 gene and/or the JAML gene; the nucleic acid amplification technique comprises amplifying the BRCA1 gene and/or the JAML gene using a pair of primers.

4. The method of claim 1, wherein expressions of the BRCA1 gene and/or the JAML gene are detected using an immunization means.

5. The method of claim 4, wherein the expressions of the BRCA1 gene and/or the JAML gene are detected using an ELISA detection kit and/or a colloidal gold detection kit.

6. A method of selecting and breeding pigs, the method comprising detecting a BRCA1 gene and/or a JAML gene, or an expression product thereof.

7. The method of claim 6, wherein expressions of the BRCA1 gene and/or the JAML gene are detected using a sequencing technique, a nucleic acid hybridization technique, or a nucleic acid amplification technique.

8. The method of claim 7, wherein the nucleic acid hybridization technique comprises employing a probe adapted to hybridize to nucleic acid sequences of the BRCA1 and/or JAML genes; the nucleic acid amplification technique comprises employing a pair of primers to amplify the BRCA1 and/or JAML genes.

9. The method of claim 6, wherein expressions of the BRCA1 gene and/or the JAML gene are detected using an immunization means.

10. The method of claim 9, wherein the expressions of the BRCA1 gene and/or the JAML gene are detected using an ELISA detection kit and/or a colloidal gold detection kit.

11. A long non-coding RNA (lncRNA) XLOC_011001, having more than 90% sequence homology with SEQ ID NO. 1.

12. The lncRNA of claim 11, wherein the lncRNA has more than 95% sequence homology with SEQ ID NO. 1.

13. The lncRNA of claim 11, wherein the lncRNA has a sequence shown in SEQ ID NO. 1.

14. A method of using the long non-coding RNA (lncRNA) of claim 11 in predicting variation of porcine intramuscular fat profile, in selecting and breeding pigs, or in preparation of a kit adapted to detect variation of porcine intramuscular fat profile, the method comprising detecting the expression of the lncRNA.

15. A kit adapted to detect variation of porcine intramuscular fat profile, the kit comprising a pair of primers for nucleic acid amplification, and the sequences of the pair of primers are represented by SEQ ID NO. 2 and SEQ ID NO. 3.

16. A method of using the kit of claim 15 in predicting variation of porcine intramuscular fat profile or in selecting and breeding pigs, the method comprising detecting a BRCA1 gene and/or a JAML gene, or an expression product thereof.
